(19) United States
(12) Patent Application Publication
(10) Pub. No.: US 2021/0164016 A1
(43) Pub. Date: Jun. 3, 2021

(54) SINGLE MOLECULE NUCLEIC ACID DETECTION BY MISMATCH CLEAVAGE

(71) Applicant: STRATOS GENOMICS INC., Seattle, WA (US)

(72) Inventors: Mark Stamatios Kokoris, Bothell, WA (US); John Tabone, Kirkland, WA (US); Melud Nabavi, Seattle, WA (US); Cara Machacek, Seattle, WA (US)

(21) Appl. No.: 16/636,619

(22) PCT Filed: Aug. 3, 2018
(86) PCT No.: PCT/US18/45181

§ 371 Date: Feb. 4, 2020

Related U.S. Application Data
(60) Provisional application No. 62/541,285, filed on Aug. 4, 2017.

(51) Int. Cl. C12Q 1/683

(52) U.S. Cl. CPC C12Q 1/683; C12Q 2521/301; C12Q 2565/631; C12Q 2563/179; C12Q 2565/1025; C12Q 2537/113

(57) ABSTRACT

Methods and materials are provided for detecting nucleic acid sequence differences including single nucleotide mutations or polymorphisms, one or more nucleotide insertions, and one or more nucleotide deletions in single molecule target members present in a test population of nucleic acid fragments. Heteroduplexes are formed between members of the test nucleic acid population and their corresponding complements provided in a pool of mismatch cleavage probes. Mismatched base pairs in the heteroduplexes are specifically cleaved and cleaved probe fragments are electronically detected to signal the present of the target members in the test population.

### FIELD OF THE INVENTION

This invention is related to materials and methods for the detection of mutations or polymorphisms in target nucleic acids at the single molecule level. More specifically, the invention provides novel mismatch cleavage probes and methods of use that facilitate the genetic screening of hereditary diseases, cancer, and infectious agents. The methods are also useful for the detection of genetic polymorphisms.

### BACKGROUND OF THE INVENTION

There is a great need in both basic and clinical research to identify DNA sequence variations with high efficiency and accuracy. The current techniques for detection of such variation can be divided into two groups: 1) detection of known mutations or polymorphisms and 2) detection of unknown mutations or polymorphisms (also referred to as mutation scanning). A variety of methods have been developed for detecting mutations and polymorphisms and include techniques such as direct DNA sequencing, allele-specific oligonucleotide hybridization, digital PCR, allele-specific PCR, DNA arrays, and PCR/LDR. Of these, next-generation DNA sequencing (NGS) has been heralded as having the potential to revolutionize and make feasible the field of personalized medicine. Indeed, it is now possible to sequence billions of nucleotides and to identify inherited clonal mutations. However, such direct DNA sequencing approaches are laborious and expensive and at present are not practical solutions for routine diagnostic screening. In addition, all NGS methods, as well most other molecular approaches, have a relatively high error rate due to, e.g., mutations introduced during PCR by DNA polymerase misincorporations and thus fail to provide efficient and accurate platforms for personalized medicine.

Technologies to sequence DNA at the single molecule level have been anticipated to resolve most, if not all, of the above problems. Importantly, single molecule sequencing eliminates the error-prone amplification step during sample preparation. One single molecule sequencing strategy that has generated much interest to date is based on the use of nanopores. The basic concept of nanopore sequencing is to pass a single-stranded DNA molecule through a nanoscale pore embedded in a membrane and measure the ensuing changes in ion current passing through the pore. In theory, individual bases induce characteristic electronic signals as they pass through the narrowest constriction of the pore, generating nucleotide-specific signals. The head-to-tail sequential feed-through of DNA should allow for unlimited read length without complicated amplification or labeling steps. In practice, nanopore-based sequencing has been hampered by the fast translocation speed of DNA through nanopores together with the fact that several nucleotides contribute to the recorded signals in the most developed systems, limiting resolution of the read-out and preventing single base calling. To date, nanopore-based DNA sequencing has not offered a practical approach to routine screening for genetic mutations or polymorphisms.

U.S. Pat. No. 6,465,193 to Akeson et al. discloses targeted molecular bar codes that are capable of producing signals upon translocation through a nanopore and their use the detection of analytes of interest. The target molecular bar codes are comprised of a signal-generating bar code linked to a binding pair member, which may be any moiety capable of interacting with the analyte of interest, e.g., a nucleic acid or an oligonucleotide. Linkage is preferably mediated by a cleavable linkage group that functions to release the molecular bar code from the binding pair member following analyte binding. The detection methods disclosed in the '193 patent involve the following steps: binding of the target analyte to the targeted molecular bar code; separation of the unbound targeted molecular bar code fraction from the bound fraction; cleavage of the linkage group of the bound targeted molecular bar code to release the molecular bar code; and electronic detection of the molecular bar code in a nanopore. The step of separating the unbound targeted molecular bar code fraction from the bound fraction is thus critical to the accuracy of the method and places strenuous demands on the quality of the purification/separation scheme. The '193 patent discloses that purification can be facilitated, e.g., by binding the target sequence to a solid support. This approach has the disadvantage of introducing a complicated sample prep step that precludes, e.g., straightforward multiplexing of the detection assay.

Thus, there is a need in the art for new methodologies with the sensitivity, specificity, and scalability to detect panels, not only of clonal, or inherited, mutations, but also of very low frequency genetic alterations, such as subclonal and random mutations, so as to enable the comprehensive study of heterogeneous populations that characterize most biological samples.

### BRIEF SUMMARY

The invention is generally directed to methods and materials for single molecule detection of target nucleic acids based on cleavage of mismatched bases between a target nucleic acid and a mismatch cleavage probe that provides target identifier moieties capable of generating distinct and reproducibly detectable signals. In one aspect, the invention provides a method for determining at least one mutation or a polymorphism in a single molecule target sequence of a polynucleotide relative to a reference sequence of the polynucleotide including the steps of: (a) providing a test sample comprising a plurality of single-stranded polynucleotides; (b) providing a mismatch cleavage probe including: i. an oligonucleotide, wherein the oligonucleotide includes a reference sequence, wherein the reference sequence includes a sequence of the reverse complement of the single-stranded target nucleic acid and contains one or more nucleotide differences relative to the target nucleic acid, wherein the oligonucleotide is capable of hybridizing to the target nucleic acid to form a heteroduplex, wherein the heteroduplex comprises one or more base pair mismatches; ii. a first target identifier linked to the oligonucleotide 5′ to the position of the one or more nucleotide differences; and iii. a second target identifier linked to the oligonucleotide 3′ to the position of the one more nucleotide differences; wherein the first and second target identifiers are capable of generating distinct and reproducibly detectable signals; (c) mixing the test sample with the mismatch cleavage probe under annealing conditions to form heteroduplexes between the mismatch cleavage probe and the target sequence; (d) contacting the heteroduplexes with a cleavage factor, wherein the cleavage factor is capable of cleaving mismatched bases in the heteroduplexes, wherein cleavage of the heteroduplex dissociates the first and second target identifiers of the mismatch cleavage probe; (e) optionally providing conditions to denature the heteroduplexes; and (f) determining the presence of the cleaved target sequence by detecting the dissociation of the first and second target identifiers.

In some embodiments, the cleavage factor is an endonuclease. In other embodiments, the test sample is cell-free DNA. In other embodiments, the method is multiplexed by providing a plurality of pooled mismatched cleavage probes in step (b) to determine at least one mutation in a plurality of target sequences. In some embodiments, the plurality of target sequences includes a plurality of biomarkers, target sequences from a plurality of test subjects, or a plurality of fragments including the entire sequence of one or more test genes. In other embodiments, the method further includes a polishing step to reduce the concentration of damaged nucleic acids in the test sample damage prior to the step of mixing the test sample with the mismatch cleavage probe or to reduce the concentration of damaged mismatch cleavage probes prior to the step of mixing the test sample with the mismatch cleavage probe. In other embodiments, the method further includes a step to isolate the heteroduplexes by binding to an immobilized MutS protein prior to the step of contacting the heteroduplexes with a mismatch endonuclease. In yet other embodiments, the method further includes a step to optimize conditions for mismatch cleavage prior to the step of contacting the heteroduplexes with the endonuclease. In some embodiments, the endonuclease is a variant engineered to increase specificity for mismatched base pairs. In other embodiments, the mismatch cleavage probe includes at least one duplex stabilizer moiety at an end of the reference oligonucleotide. In other embodiments, the step of determining the presence of the cleaved target sequence comprises passage of the cleaved mismatch cleavage probes through a nanopore to generate electronic signals. In yet other embodiments, the methods further includes one or more controls including positive controls, negative controls, and process controls.

In another aspect, the invention provides a mismatch cleavage probe for detecting single molecule single-stranded target nucleic acid in a sample including: (a) an oligonucleotide, wherein the oligonucleotide includes a reference sequence, wherein the reference sequence includes a sequence of the reverse complement of the single-stranded target nucleic acid and contains one or more nucleotide differences relative to the target nucleic acid, wherein the oligonucleotide is capable of hybridizing to the target nucleic acid to form a heteroduplex, wherein the heteroduplex includes one or more base pair mismatches; (b) a first target identifier linked to the oligonucleotide 5′ to the position of the one or more nucleotide differences, and (c) a second target identifier linked to the oligonucleotide 3′ to the position of the one more nucleotide differences; wherein the first and second target identifiers are capable of generating distinct and reproducibly detectable signals. In some embodiments, the distinct and reproducibly detectable signals are electronic. In some embodiments, the first and second target identifiers includes translocation control elements. In other embodiments, the mismatch cleavage probe further includes a hydrophobic capture element and a leader sequence associated with the first target identifier and a biotin moiety associated with the second target identifier. In yet other embodiments, the mismatch cleavage probe further includes a first hydrophobic capture element and a first leader sequence associated with the first target identifier and a second hydrophobic capture element and a second leader sequence associated with the second target identifier. In other embodiments, the target identifiers include a plurality of unique codes, wherein each individual code is associated with a translocation control element. In some embodiments, the target identifiers include from around 2 to around 10 codes. In yet other embodiments, the sequence of the each code is selected from the group including: DDXXXXXXX, DDDD88XDL, L8DX88DDDD, and 8DX8888DDDD, wherein D is PEG-6, X is PEG-3, 8 is reverse amidite T, and L is C2. In some embodiments, the mismatch cleavage probes further includes a duplex stabilizer associated with at least one end of the reference oligonucleotide that in certain embodiments may be a spermine or a G-clamp moiety. In yet other embodiments, the sequence of the reference oligonucleotide includes the wild-type allele of a tumor biomarker or a sequence from a pathogenic microorganism.

In another aspect, the invention provides a circular mismatch cleavage probe for detecting single molecule target nucleic acid in a sample including: (a) an oligonucleotide, wherein the oligonucleotide includes a reference sequence, wherein the reference sequence includes a sequence of the reverse complement of the single-stranded target nucleic acid and contains one or more nucleotide differences relative to the target nucleic acid, wherein the oligonucleotide is capable of hybridizing to the target nucleic acid to form a heteroduplex, wherein the heteroduplex includes one or more base pair mismatches; (b) a target identifier linked to the 5′ end of the oligonucleotide, wherein the target identifier includes a translocation control element and wherein the target identifier is capable of generating a distinct and reproducibly detectable signal upon passage through a nanopore; and (c) a leader sequence associated with a hydrophobic capture element, wherein the hydrophobic capture element is linked to the target identifier and the leader sequence is linked to the 3′ end of the oligonucleotide; wherein the circular mismatched cleavage probe is not capable of passage through a nanopore, wherein cleavage of the oligonucleotide linearizes the mismatch cleavage probe, and wherein the linear mismatch cleavage probe is capable of passage through a nanopore.

In another aspect, the invention provides a method for amplifying a signal indicating at least one mutation or a polymorphism in a target sequence of a polynucleotide relative to a reference sequence of the polynucleotide including: (a) a mismatch cleavage stage, wherein the mismatch cleavage stage includes contacting the target sequence with a mismatch amplifier probe and a mismatch endonuclease to produce a cleaved amplifier probe and (b) iterative rounds of a signal amplification stage, wherein a single round of the signal amplification stage includes contacting the amplifier probe with a pool of amplification code probes and a nickase enzyme to produce a cleaved amplification code probe capable of producing a distinct and reproducible signal upon passage through a nanopore. In some embodiments, the mismatch cleavage stage includes the steps of: (a) providing a test sample including a plurality of denatured polynucleotides; (b) providing a mismatch amplifier probe including a reference oligonucleotide, a first hybridization oligonucleotide, and first nickase recognition oligonucleotide, and a biotin moiety; (c) mixing the test sample with the mismatch amplifier probe under annealing conditions to form heteroduplexes between the mismatch amplifier probe and a target sequence; (d) contacting the heteroduplexes with an endonuclease capable of cleaving mismatched bases in the heteroduplex, wherein cleavage of the heteroduplex releases an amplifier probe comprising the first hybridization oligonucleotide and the first nickase recognition oligonucleotide; and (e) removing the biotin moiety and associated nucleic acids from the test sample. In other embodiments, the signal amplification stage includes the steps of: (f) providing a pool of amplification code probes, wherein the amplification code probes includes a second hybridization oligonucleotide, a second nickase recognition oligonucleotide, a target identifier, a hydrophobic capture element, a leader sequence, and a streptavidin moiety; (g) providing conditions to hybridize the amplification code probes of step (d) to the amplifier probe of claim 28 to form a double-stranded nucleic acid comprising a double-stranded nickase site; (h) contacting the double-stranded nickase site with a nickase endonuclease to cleave the second nickase recognition oligonucleotide and release a cleaved amplification code probe; (i) heating the sample to release the uncleaved amplifier probe; and (J) recycling the amplifier probe a plurality of times through steps (g) through (i) to provide a plurality of cleaved amplification code probes.

In another aspect, the invention provides a mismatch amplifier probe for amplifying a signal indicating at least one mutation or a polymorphism in a target sequence of a polynucleotide relative to a reference sequence of the polynucleotide including: (a) an oligonucleotide, wherein the oligonucleotide includes a reference sequence, wherein the reference sequence includes a sequence of the reverse complement of the single-stranded target nucleic acid and contains one or more nucleotide differences relative to the target nucleic acid, wherein the oligonucleotide is capable of hybridizing to the target nucleic acid to form a heteroduplex, wherein the heteroduplex includes one or more base pair mismatches; (b) a first hybridization oligonucleotide; (c) a first nickase recognition oligonucleotide; and (d) a biotin moiety.

In another aspect, the invention provides an amplification code probe for amplifying a signal indicating at least one mutation or a polymorphism in a target sequence of a polynucleotide relative to a reference sequence of the polynucleotide including: (a) second hybridization oligonucleotide, wherein the sequence of the second hybridization oligonucleotide includes the reverse complement of the sequence of the first hybridization oligonucleotide; (b) a second nickase recognition oligonucleotide, wherein the sequence of the second nickase recognition oligonucleotide includes the reverse complement of the sequence of the first nickase recognition oligonucleotide, and wherein the second nickase recognition oligonucleotide is capable of being cleaved by a nickase endonuclease; (c) a target identifier; (d) a hydrophobic capture element; (e) a leader sequence; and (f) a streptavidin moiety.

In another aspect, the invention provides a circular amplification code probe for amplifying a signal indicating at least one mutation or a polymorphism in a target sequence of a polynucleotide relative to a reference sequence of the polynucleotide including: (a) a second hybridization oligonucleotide, wherein the sequence of the second hybridization oligonucleotide includes the reverse complement of the sequence of the first hybridization oligonucleotide; (b) a second nickase recognition oligonucleotide linked to the 3′ end of the second hybridization oligonucleotide, wherein the sequence of the second nickase recognition oligonucleotide includes the reverse complement of the sequence of the first nickase recognition oligonucleotide, and wherein the second nickase recognition oligonucleotide is capable of being cleaved by a nickase endonuclease; (c) a target identifier linked to the 5′ end of the second hybridization oligonucleotide; (d) a hydrophobic capture element linked to the 5′ end of the target identifier; and (e) a leader sequence linked to the 5′ end of the hydrophobic capture element and the 3′ end of the second nickase recognition oligonucleotide.

### BRIEF DESCRIPTION OF THE FIGURES

In the figures, the sizes and relative positions of elements are not necessarily drawn to scale and some of these elements are arbitrarily enlarged and positioned to improve figure legibility. Further, the particular shapes of the elements as drawn are not intended to convey any information regarding the actual shape of the particular elements, and have been solely selected for ease of recognition in the figures.

FIG. 1 shows a cartoon illustrating one method of the invention for using mismatch cleavage to detect the presence of a target sequence of interest in a complex sample.

FIGS. 2A and 2B show cartoons of one embodiment of a mismatch cleavage probe of the present invention in uncleaved and cleaved configurations, respectively.

FIGS. 3A and 3B show cartoons of one embodiment of a symmetrical mismatch cleavage probe of the present invention in uncleaved and cleaved configurations, respectively.

FIGS. 4A and 4B show cartoons of one embodiment of a circular mismatch cleavage probe of the present invention in uncleaved and cleaved configurations, respectively.

FIG. 5 shows certain embodiments of reporter codes incorporated into the mismatch cleavage probes of the invention.

FIG. 6 shows one embodiment of a target identifier incorporated into the mismatch cleavage probes of the invention.

FIG. 7 shows a cartoon illustrating a first stage of one method of the invention for amplifying the signal from mismatch cleavage using a mismatch amplifier probe.

FIG. 8 shows a cartoon illustrating a second stage of one method of the invention for amplifying the signal from mismatch cleavage using an amplification code probe.

FIGS. 9A and 9B show cartoons of one embodiment of a circular amplification code probe of the invention in uncleaved and cleaved configurations, respectively.

FIG. 10 shows resistance to endonuclease-mediated cleavage by a perfectly paired homoduplex, and cleavage of a heteroduplex sample.

FIGS. 11A and 11B show time traces for recorded current measurements caused by different probes passing through a nanopore.

### DEFINITIONS

As used herein, the singular forms “a”, “an” and “the” are intended to include the plural forms as well, unless the context clearly indicates otherwise. Additionally, the use of “or” is intended to include “and/or” unless the context clearly indicates otherwise.

The term “isolated nucleic acid” refers to a DNA or RNA molecule that is separated from sequences with which it is normally immediately contiguous (in the 5′ and 3′ directions) in the naturally occurring genome of the organism in which it originates. The term “isolated nucleic acid” also includes a nucleic acid which exists as a separate molecule independent of other nucleic acids such as a nucleic acid fragment produced by chemical means or restriction endonuclease treatment.

A test nucleic acid or target nucleic acid, as used herein, is DNA or RNA, each of which bears at least one mutation or polymorphism relative to a reference nucleic acid. In certain embodiments, the target nucleic acid is present in cell-free DNA (cfDNA) or circulating tumor DNA (ctDNA) and will be from around 100 to around 200 nucleotides in length.

As used herein, the term “reference sequence” typically refer to the nucleic acid molecule or polynucleotide having a sequence prevalent in the general population that is not associated with any disease or discernible disease phenotype. It is noted that in the general population, wild-type genes may include multiple prevalent versions that contain alterations in sequence relative to each other and yet do not cause a discernible pathological effect. These variations are designated “polymorphisms” or “allelic variations.” It is therefore possible that a reference sequence is a mixture of the most common polymorphisms. Alternatively, one reference sequence may be used that has been selected for its particular sequence. In other embodiments, the reference sequence may include part of a foreign genetic sequence e.g. the genome of an invading microorganism. Non-limiting examples include bacteria and their phages, viruses, fungi, protozoa, mycoplasms, and the like. In some embodiments the reference sequence may be the sequence of bacterial 16S rRNA or 23S rRNA.

The term “oligonucleotide” as used herein includes linear oligomers of natural or modified monomers or linkages, including deoxyribonucleosides, ribonucleosides, and the like, capable of specifically binding to a target polynucleotide by way of a regular pattern of monomer-to-monomer interactions, such as Watson-Crick type of base pairing, base stacking, Hoogsteen or reverse Hoogsteen types of base pairing, or the like. Usually monomers are linked by phosphodiester bonds or analogs thereof to form oligonucleotides ranging in size from a few monomeric units, e.g. 3-4, to several tens of monomeric units, e.g. 40-60. Whenever an oligonucleotide is represented by a sequence of letters, such as “ATGCCTG.” it will be understood that the nucleotides are in 5′ 3′ order from left to right and that “A” denotes deoxyadenosine. “C” denotes deoxycytidine, “G” denotes deoxyguanosine, “T” denotes thymidine, and “U” denotes uridine, unless otherwise noted. The term “dNTP” is an abbreviation for “a deoxyribonucleoside triphosphate,” and “dATP”, “dCTP”, “dGTP”, “dTTP”, and “dUTP” represent the triphosphate derivatives of the individual deoxyribonucleosides. Usually oligonucleotides comprise the natural nucleotides; however, they may also comprise non-natural nucleotide analogs. It is clear to those skilled in the art when oligonucleotides having natural or non-natural nucleotides may be employed, e.g. where processing by enzymes is called for, usually oligonucleotides consisting of natural nucleotides are required.

A “mutation,” as used herein, refers to a nucleotide sequence change (i.e., a single or multiple nucleotide substitution, deletion, or insertion) in a nucleic acid sequence that produces a phenotypic result. A nucleotide sequence change that does not produce a detectable phenotypic result is referred to herein as a “polymorphism.” “Homologous,” as used herein in reference to nucleic acids, refers to the nucleotide sequence similarity between two nucleic acids. When a first nucleotide sequence is identical to a second nucleotide sequence, then the first and second nucleotide sequences are 100% homologous. The homology between any two nucleic acids is a direct function of the number of matching nucleotides at a given position in the sequence, e.g., if half of the total number of nucleotides in two nucleic acids are the same then they are 50% homologous. In the present invention, an isolated test nucleic acid and a control nucleic acid are at least 90% homologous. Preferably, an isolated test nucleic acid and a control nucleic acid are at least 95% homologous, more preferably at least 99% homologous.

The term “complementary” refers to two nucleic acid strands that exhibit substantial normal base pairing characteristics. Complementary nucleic acid strands contain a series of consecutive nucleotides which are capable of forming base pairs to produce a region of double-strandedness. This region is referred to as a duplex. A duplex may be either a homoduplex or a heteroduplex that forms between nucleic acids because of the orientation of the nucleotides on the RNA or DNA strands; certain bases attract and bond to each other to form multiple Watson-Crick base pairs. Thus, adenine in one strand of DNA or RNA, pairs with thymine in an opposing complementary DNA strand, or with uracil in an opposing complementary RNA strand. Guanine in one strand of DNA or RNA, pairs with cytosine in an opposing complementary strand. By the term “heteroduplex” is meant a structure formed between two annealed, complementary, and homologous nucleic acid strands (e.g. an annealed isolated test and control nucleic acid) in which one or more nucleotides in the first strand is unable to appropriately base pair with the second opposing, complementary and homologous nucleic acid strand because of one or more mutations. Examples of different types of heteroduplexes include those which exhibit a point mutation (i.e. bubble), insertion or deletion mutation (i.e. bulge).

As used herein, the term “annealing” refers to the formation of at least partially double stranded nucleic acid by hybridization of at least partially complementary nucleotide sequences. A partially double stranded nucleic acid can be due to the hybridization of a smaller nucleic acid strand to a longer nucleic acid strand, where the smaller nucleic acid is 100% identical to a portion of the larger nucleic acid. A partially double stranded nucleic acid can also be due to the hybridization of two nucleic acid strands that do not share 100% identity but have sufficient homology to hybridize under a particular set of hybridization conditions. The term “hybridization” refers to the hydrogen bonding that occurs between two complementary nucleic acid strands.

As used herein, the phrase “preferentially hybridizes” refers to a nucleic acid strand which anneals to and forms a stable duplex, either a homoduplex or a heteroduplex, under normal hybridization conditions with a second complementary nucleic acid strand, and which does not form a stable duplex with unrelated nucleic acid molecules under the same normal hybridization conditions. The formation of a duplex is accomplished by annealing two complementary nucleic acid strands in a hybridization reaction. The hybridization reaction can be made to be highly specific by adjustment of the hybridization conditions (often referred to as hybridization stringency) under which the hybridization reaction takes place, such that hybridization between two nucleic acid strands will not form a stable duplex, e.g., a duplex that retains a region of double-strandedness under normal stringency conditions, unless the two nucleic acid strands contain a certain number of nucleotides in specific sequences which are substantially or completely complementary. “Normal hybridization or normal stringency conditions” are readily determined for any given hybridization reaction (see, for example, Ausubel et al., Current Protocols in Molecular Biology, John Wiley & Sons, Inc., New York, or Sambrook et al., Molecular Cloning: A Laboratory Manual, Cold Spring Harbor Laboratory Press).

The term “denaturing” or “denatured,” when used in reference to nucleic acids, refers to the conversion of a double stranded nucleic acid to a single stranded nucleic acid. Methods of denaturing double stranded nucleic acids are well known to those skilled in the art, and include, for example, addition of agents that destabilize base-pairing, increasing temperature, decreasing salt, or combinations thereof. These factors are applied according to the complementarity of the strands, that is, whether the strands are 100% complementary or have one or more non-complementary nucleotides.

As used herein a “mismatch” can be the result of two non-complementary bases occurring opposite each other. A mismatch site can consist of a cluster of any number of unpaired nucleotides, including nucleotide base-pairs that are made unstable by neighboring mismatches. A mismatch can also be the result of one or more bases occurring on one strand that do not have a numerical opposite on the opposite strand. For example, at the site of a mismatch there might be 1 unpaired base on one strand and no unpaired bases on the other strand. This would result in a site of sequence length heterogeneity in which a single unpaired nucleotide is contained in one strand at that site.

The term “base pair mismatch” indicates a base pair combination that generally does not form in nucleic acids according to Watson and Crick base pairing rules. For example, when dealing with the bases commonly found in DNA, namely adenine, guanine, cytosine and thymidine, base pair mismatches are those base combinations other than the A-T and G-C pairs normally found in DNA. As described herein, a mismatch may be indicated, for example as C/C meaning that a cytosine residue is found opposite another cytosine, as opposed to the proper pairing partner, guanine. C>T indicates the substitution of a cytosine residue for a thymidine residue giving rise to a mismatch. Inappropriate substitution of any base for another giving rise to a mismatch or a polymorphism may be indicated this way.

The phrase “DNA insertion or deletion” refers to the presence or absence of “matched” bases between two strands of DNA such that complementarity is not maintained over the region of inserted or deleted bases.

The phrase “flanking nucleic acid sequences” refers to those contiguous nucleic acid sequences that are 5′ and 3′ to the endonuclease cleavage site.

The term “cleaving” means digesting the polynucleotide with enzymes or otherwise breaking phosphodiester bonds within the polynucleotide. As used herein, the term “strand cleavage activity” or “cleavage” refers to the breaking of a phosphodiester bond in the backbone of the polynucleotide strand, as in forming a nick. Strand cleavage activity can be provided by an endonuclease.

The term “mismatch cleavage endonuclease” refers to an enzyme that recognizes mismatched bases in polynucleotide heteroduplexes and causes cleavage of at least one strand of the mismatch. Non-limiting examples of such endonuclease include single-strand specific nucleases, such as CEL I (Till et al., Nuc. Acid Res. 32(8):2632-2641 (2004)) and CEL II (U.S. Pat. No. 7,129,075), bacteriophage resolvases, such as T7 endonuclease I and T4 endonucleases VII (Mashal, et al., Nature Genetics 9:177-183 (1995)), E. coli Endonuclease V (Yao and Kow, J. Biol. Chem. 272(49): 30774-30779 (1997)), and Archaeal TkoEndoMS (Ishino et al., Nuc. Acids Res. 44(7):2977-2986 (2016)). The methods of the present invention include combinations of mismatch cleavage endonucleases demonstrating the following properties: the ability to detect all mismatches, whether known or unknown between hybridized polynucleotides, the ability to detect mismatches over a pH range of 5-9, the ability to exhibit substantial activity over the entire pH range; the ability to recognize polynucleotide loops and insertions in hybridized polynucleotides; the ability to catalyze formation of a substantially single-stranded nick at the heteroduplex site containing a mismatch; the ability to recognize a mutation in a target polynucleotide sequence, without being substantially affected by flanking DNA sequences. Mismatch cleavage endonucleases of the present invention may also include variant endonucleases engineered to display improved properties, e.g., improved substrate specificity.

The term “multiplex analysis” refers to the simultaneous assay using a pool of different mismatch cleavage probes and/or of pooled of different nucleic acid samples according to the methods described herein.

As used herein, the term “test sample” refers to anything which may contain a target nucleic acid for which detection assay is desired. In many cases, the nucleic acid is a cell-free (cf) nucleic acid molecule, such as a circulating tumor (ct) DNA molecule encoding all or part of a cancer biomarker. The sample may be a biological sample, such as a biological fluid or a biological tissue. Examples of biological fluids include urine, blood, plasma, serum, saliva, semen, stool, sputum, cerebrospinal fluid, tears, mucus, amniotic fluid or the like. Biological tissues are aggregates of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

### GENERAL DESCRIPTION

The present invention is generally directed to the identification of single molecule target nucleic acid sequences in a test population that contain polymorphic sequences relative to nucleic acid sequences in a reference population. In particular, the invention is directed to methods of detecting single molecule target nucleic acids based on cleavage of mismatched bases between a target nucleic acid and a mismatch cleavage probe providing target identifier moieties capable of generating distinct and reproducibly detectable signals, e.g., electronic signals detectable by passage through a nanopore. By enabling detection at the single molecule level, the present invention offers considerable advantages over known nucleic acid detection methods and systems requiring target amplification and sequencing, which are time consuming and generate less reliable and lower signals. An important feature of the present invention is the ability to multiplex the analysis, i.e., to detect large populations of target nucleic acids in a single test sample or a mixture of a plurality of test samples.

As further discussed below, mismatch cleavage probes of the present invention are designed to include a reference oligonucleotide capable of hybridizing to a target nucleic acid to form a heteroduplex containing at least one base pair mismatch. Each probe also includes a 5′ specific target identifier moiety and a 3′ specific target identifier moiety, positioned 5′ and 3′ to the base pair mismatches, respectively. Advantageously, heteroduplexes are specifically cleaved at the position of the base pair mismatches, either enzymatically or chemically. In certain embodiments, the heteroduplexes are cleaved by endonucleases, e.g., mismatch cleavage endonucleases. Cleavage of the mismatched bases produces a 5′ fragment, including the 5′ specific target identifier moiety, and a 3′ fragment, including the 3′ specific target identifier moiety from the original mismatch target probe. Such dissociation of the 5′ and 3′ ends of the mismatch probe indicates the presence of the target nucleic acid in the test sample and, according to the methods of the present invention, is detected by the uncoupling of the 5′ specific (or “first”) and the 3′ specific (or “second”) target identifiers.

The techniques described herein are extremely useful for detecting any biomarker of interest for medical, security, surveillance purposes, and the like. In certain embodiments, biomarkers include DNA mutations and polymorphisms associated with mammalian diseases (such as cancer and various inherited diseases), as well as mutations which facilitate the development of therapeutics for their treatment. Mutations and polymorphism associated with cancer are also be referred herein to as “cancer biomarkers” or “tumor biomarkers”. These methods are not narrowly limited to any particular gene mutations in any particular cancer, since any mutation that is associated with any cancer would be expected to be accurately monitored by these methods. Exemplary classes of cancer biomarker include tumor suppressor genes, oncogenes, and DNA replication or repair genes. Non-limiting examples of such genes include Bcl2, Mdm2, Cdc25A, Cyclin D1, Cyclin E1, Cdk4, survivin, HSP27, HSP70, p53, p21^{Cip}, p16^{Ink4a}, p19^{ARF}, p15^{INK4b}, p27^{Kip} Bax, growth factors, EGFR, Her2-neu, ErbB-3, ErbB-4, c-Met, c-Sea, Ron, c-Ret, NGFR, TrkB, TrkC, IGF1R, CSF1R, CSF2, c-Kit, AXL, Flt-1 (VEGFR-1), Flk-1 (VEGFR-2), PDGFRα, PDGFRβ, FGFR-1, FGFR-2, FGFR-3, FGFR-4, other protein tyrosine kinase receptors, β-catenin, Wnt(s), Akt, Tcf4, c-Myc, n-Myc, Wisp-1, Wisp-3, K-ras, H-ras, N-ras, c-Jun, c-Fos, PI3K, c-Src, Shc, Raf1, TGFβ, and MEK, E-Cadherin, APC, TβRII, Smad2, Smad4, Smad 7, PTEN, VHL, BRCA1, BRCA2, ATM, hMSH2, hMLH1, hPMS1, hPMS2, and hMSH3.

Non-limiting examples of cancer include adrenal cortical cancer, anal cancer, bile duct cancer, bladder cancer, bone cancer, brain or a nervous system cancer, breast cancer, cervical cancer, colon cancer, rectal cancer, colorectal cancer, endometrial cancer, esophageal cancer, Ewing family of tumor, eye cancer, gallbladder cancer, gastrointestinal carcinoid cancer, gastrointestinal stromal cancer, Hodgkin Disease, intestinal cancer, Kaposi Sarcoma, kidney cancer, large intestine cancer, laryngeal cancer, hypopharyngeal cancer, laryngeal and hypopharyngeal cancer, leukemia, acute lymphocytic leukemia (ALL), acute myeloid leukemia (AML), chronic lymphocytic leukemia (CLL), chronic myeloid leukemia (CIVIL), chronic myelomonocytic leukemia (CMML), non-HCL lymphoid malignancy (hairy cell variant, splenic marginal zone lymphoma (SMZL), splenic diffuse red pulp small B-cell lymphoma (SDRPSBCL), chronic lymphocytic leukemia (CLL), prolymphocytic leukemia, low grade lymphoma, systemic mastocytosis, or splenic lymphoma/leukemia unclassifiable (SLLU)), liver cancer, lung cancer, non-small cell lung cancer, small cell lung cancer, lung carcinoid tumor, lymphoma, lymphoma of the skin, malignant mesothelioma, multiple myeloma, nasal cavity cancer, paranasal sinus cancer, nasal cavity and paranasal sinus cancer, nasopharyngeal cancer, neuroblastoma, non-Hodgkin lymphoma, oral cavity cancer, oropharyngeal cancer, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma, adult soft tissue sarcoma, skin cancer, basal cell skin cancer, squamous cell skin cancer, basal and squamous cell skin cancer, melanoma, stomach cancer, small intestine cancer, testicular cancer, thymus cancer, thyroid cancer, uterine sarcoma, uterine cancer, vaginal cancer, vulvar cancer, Waldenstrom Macroglobulinemia, and Wilms Tumor.

Alternatively, the methods are also useful for forensic applications or the identification of useful traits in commercial (for example, agricultural) species.

The methods of the present invention may also be used for rapid typing of bacterial and viral strains. By “type” is meant to characterize an isogeneic bacterial or viral strain by detecting one or more nucleic acid mutations that distinguishes the particular strain from other strains of the same or related bacteria or virus. Other examples of test DNAs of particular interest for typing include test DNAs isolated from viruses of the family Retroviridae, for example, the human T-lymphocyte viruses or human immunodeficiency viruses (in particular, any one of HTLV-I, HTLV-II, HIV-1, or HIV-2), DNA viruses of the family Adenoviridae, Papovaviridae, or Herpetoviridae, bacteria, or other organisms, for example, organisms of the order Spirochaetales, of the genus Treponema or Borrelia, of the order Kinetoplastida, of the species Trypanosoma cruzi, of the order Actinomycetales, of the family Mycobacteriaceae, of the species Mycobacterium tuberculosis, or of the genus Streptococcus. The present methods are particularly applicable when it is desired to distinguish between different variants or strains of a microorganism in order to choose appropriate therapeutic interventions.

The methods of the present invention may also be used to diagnose a pathogenic bacterial infection by detecting the presence of a specific bacterial 16S rRNA gene fragment in a test sample.

### Nucleic Acid Detection by Mismatch Cleavage

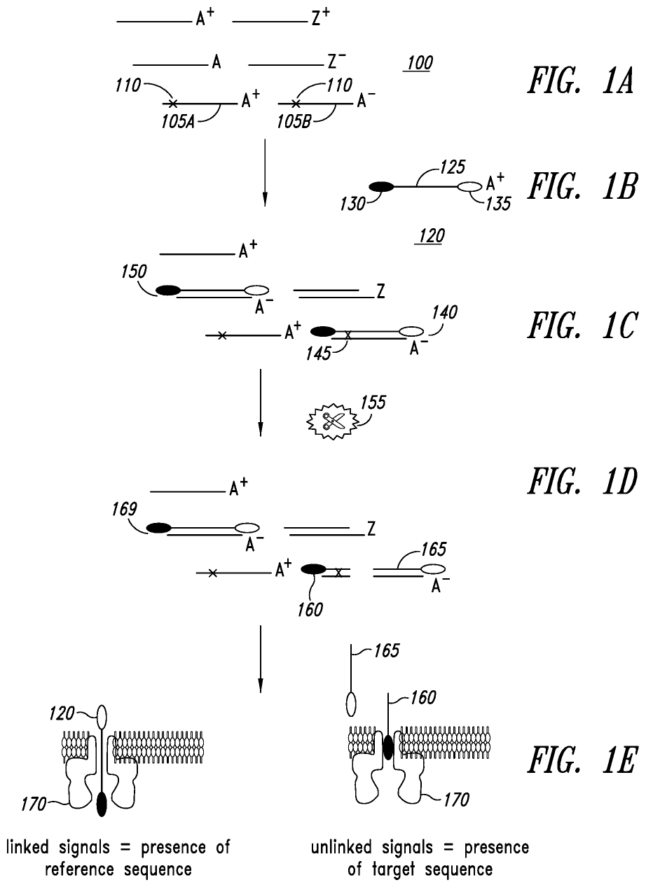

FIG. 1 is a cartoon outline of a generalized method of detecting a single molecule of a target nucleic acid of the present invention. For clarity of discussion, features illustrated in the figure are simplified and not shown to scale. In this embodiment, the sequence of the target nucleic acid has a single base pair change relative to a reference sequence. In step A of the method, test sample 100 is provided that contains a mixture of single-stranded nucleic acids. The single-stranded nucleic acids may be denatured DNA molecules or, in other embodiments, single stranded RNA molecules. Here, for simplicity, the single-stranded nucleic acids are depicted as the sense strands (+) of DNA sequence A (the wild-type version of the target sequence) and DNA sequence Z (representing a pool of non-target sequences) and the antisense strands (−) of sequences A and Z. The test sample also contains sense strand 105A and antisense strand 105B of the target nucleic acid, herein depicted as sequence A with single base pair change 110 relative to the reference sequence (e.g., the wild-type sequence) A.

The test sample may be obtained from any source, natural or synthetic, including, but not limited to, cell sources, tissue sources, or body fluid sources. Nucleic acids are extracted from the cells or body fluids using any method known in the art. The test sample may be derived from one or more individuals having a medical condition, susceptibility, or disease. In one embodiment, the test sample is a sample of cell-free DNA (cfDNA) derived from one or more individuals for detection of circulating tumor DNA (ctDNA) biomarkers. cfDNA is preferably extracted from the plasma fraction of whole blood. In one embodiment, around 10 ml of whole blood is drawn from an individual to produce around 5.5 ml of plasma, which contains around 5 to 500 ng cfDNA for analysis.

In certain embodiments, the methods of the present invention may further include at least one step to reduce the concentration of nucleic acids damaged during preparation and/or extraction of the test sample. Such sample “polishing steps” advantageously reduce the likelihood of false positives during mismatch cleavage step D. Sample polishing steps may include, e.g., pre-treatment of the test sample containing double-stranded nucleic acids with the mismatch endonuclease(s) of step D under low-stringency cleavage conditions.

A test sample of single-stranded nucleic acids may be produced in a variety of ways, including denaturation of double-stranded DNA by heating, treatment with a chaotropic solvent, and the like, using techniques well known in the art, e.g. Britten et al. Methods in Enzymology, 29: 363-418 (1974): Wetmur et al, J. Mol. Biol., 31: 349-370 (1968). In certain embodiments, denaturation of ctDNA may be accomplished by heating the DNA fragments above their Tm value (generally greater than 94° C.) for 15 seconds to 5 minutes. In other embodiments, a test sample of RNA is produced using any suitable method known in the art.

In step B of the method, mismatch cleavage probe 120 is provided that includes oligonucleotide 125, which in this embodiment comprises a sequence of the sense strand of reference sequence A. The mismatch cleavage probe also includes first target identifier 130 and second target identifier 135 that each generate a distinct and reproducible signal. As disclosed herein, the type of signals generated by the target identifiers of the present invention are not intended to be limited to any particular class and may include, e.g., electronic and fluorescent signals. Various embodiments of mismatch cleavage probe configurations are described further herein. In certain embodiments, the reference sequence may be the wild-type version of the target sequence; however, in other embodiments, the reference sequence may be a polymorphic or mutant variant of the target sequence. In other embodiments, a second mismatch cleavage probe may be used simultaneously with probe 120 in which the second probe includes a sequence of the antisense strand of the reference sequence and the same target identifiers as probe 120.

In some embodiments, a plurality of mismatch cleavage probes are provided in step B to multiplex the detection methodology. As described further herein, the combinations of codes comprising the target identifiers of the present invention provide a plurality of distinguishable signals available for multiplex analysis. In one embodiment, a multiplex detection method will include a pool of mismatch cleavage probes comprising a plurality of unique reference oligonucleotide to detect a plurality of biomarkers. In another embodiment, a multiplex detection method will include a pool of mismatch cleavage probes comprising a plurality of gene or exon fragments of a specific target gene in order to screen for unknown mutations in the target gene. In another embodiment, a multiplex detection method will simultaneously test a pool of samples derived from a plurality of individuals in which the sample from each individual is paired with a unique mismatch cleavage probe signal.

In step C of the method, the mismatch cleavage probe is mixed with the test sample under annealing conditions to allow formation of heteroduplex 140 between the mismatch cleavage probe and the target sequence, in which the heteroduplex contains at least one single base pair mismatch 145. The mismatch probe will also form homoduplex 150 with reference sequence A. The formation of duplexes under annealing conditions is also referred herein as a hybridization reaction. In some embodiments, annealing conditions generally include cooling to 45-80° C. for 2 to 60 minutes, in other embodiments, cooling to 65° C. for 15 minutes, then to room temperature for 5-30 minutes to form duplexes. In addition, the specificity of the hybridization reaction can be further controlled, e.g., by the salt concentration, under which the hybridization reaction takes place, such that hybridization between the two nucleic acid strands will not form a stable duplex, e.g., a duplex that retains a region of double-strandedness under normal stringency conditions, unless the two nucleic acid strands contain a certain number of nucleotides in specific sequences which are substantially, or completely, complementary. Thus, the phrase “preferentially hybridize” as used herein, refers to a nucleic acid strand which anneals to and forms a stable duplex, either a homoduplex or a heteroduplex, under normal hybridization conditions with a second complementary and homologous nucleic acid strand, and which does not form a stable duplex with other nucleic acid molecules under the same normal hybridization conditions. The duplexes formed in step C of the present invention are heteroduplexes when the target sequence is present in the test sample and includes a “bubble” at the region of lack of complementarity, e.g., the location of the mutation or polymorphism in the target sequence. As disclosed herein, mutations or polymorphisms may include single base changes, insertions, or deletions in the target sequence. In certain embodiments, the bubbles include from 1 to 10 unpaired bases on one or both strands of the heteroduplex. In contrast, homoduplexes are perfectly paired and do not form bubbles.

In certain embodiments, the methods of the present invention further include a step to “polish” the mismatch cleavage probe prior to step C so as to remove synthetic damage to the probe that could generate false positive signals in the detection of the target nucleic acid. In one embodiment, the mismatch cleavage probe is hybridized to a synthetic oligonucleotide including the reverse complement sequence of the reference oligonucleotide of the probe. In some embodiments, the synthetic oligonucleotide may be linked to a solid-support. Perfectly paired nucleic acids will form homoduplexes, which will be resistant to mismatch cleavage, while heteroduplexes formed due to synthetic sequence errors in the reference oligonucleotide of the mismatch probe (or in the synthetic reference oligonucleotide) will be vulnerable to mismatch cleavage. The duplexed nucleic acids are then treated with the same one or more mismatch endonucleases of step D under the same, or more stringent, cleavage reactions conditions so as to cleave heteroduplexes representative of synthetic sequence defects. Following cleavage, uncleaved homoduplexes and single-stranded uncleaved mismatch probe can be isolated from mismatch cleavage products, e.g., by chromatography.

In other embodiments, the mismatch cleavage probe may be “armored” to protect it from non-specific cleavage, e.g., by chemical modification of the phosphodiester backbone or bases at selected positions by methods known in the art. In some embodiments, artificial sequences can be added to the 5′ and/or 3′ ends of the reference oligonucleotide that include nucleotide analogs, e.g., analogs with a 2′OMe groups that are not recognized and cleaved by endonucleases.

In certain embodiments, the methods of the present invention also include at least one step to optimize the reaction conditions prior step D, e.g., to remove impurities from the test sample that may adversely affect the activity and/or specificity of the one or more mismatch endonucleases. In one embodiment, the duplexed nucleic acids are purified from the test sample following the annealing step C. Duplexed nucleic acids in which the mismatch cleavage probes includes a capture tag, e.g., a biotin moiety, may be purified by capture with streptavidin (SA) linked to a solid support. Well-known solid supports include magnetic beads or other microparticles. Also useful are polyacrylamide, glass, natural cellulose, or modified cellulose such as nitrocellulose, polystyrene, polypropylene, polyethylene, dextran, or nylon. The solid support can have virtually any structure or configuration so long as it is capable of binding to the duplexed strands. Methods of binding polynucleotide strands to solid supports are described, for example in U.S. Pat. No. 5,412,087 to McGall et al.; Shena et al. PNAS USA 93:10614-10619 (1996) and WO 95/35505. Purified duplexed nucleic acids may be added to a cleavage reaction sample in which conditions, e.g., buffer, salt, co-factors, temperature, and the like are optimized for the one or more mismatch endonucleases of step D.

In certain embodiments, the methods of the present invention include a step to purify heteroduplexed nucleic acid from homoduplexed nucleic acids prior to mismatch cleavage. In one embodiment, heteroduplexes are specifically captured from a test sample by immobilized MutS, e.g., by MutS protein immobilized on a cellulose support.

In step D of the method, the mixture is treated with at least one cleavage factor 155 that is capable of cleaving the mismatched base pair in the heteroduplex. Suitable cleavage factors will depend upon the type of heteroduplex formed in step C, which, in this embodiment, includes mismatch endonucleases. In other embodiments, suitable cleavage factors may include other enzymes, such as RNases, or chemical treatments. Cleavage of the heteroduplex generates two double-stranded products, first mismatch probe fragment 160 and the second mismatch probe fragment 165. In this manner, the first and second target identifiers of the mismatch cleavage probe are uncoupled. In contrast, homoduplex 150, comprised of the perfectly paired reference oligonucleotide and wild-type allele of the target sequence, is not cleaved; thus the first and second target identifiers of the mismatch probe remain physically associated, i.e. “coupled” or “linked”. A key feature of the methods of the invention is the reliable specificity of mismatch cleavage, and several methods and compositions are disclosed herein to enhance the specificity of mismatch cleavage by endonucleases and/or decrease nonspecific duplex cleavage. In one embodiment, the one or more mismatch endonucleases are engineered variants and have been selected for enhanced specificity. In another embodiment, the 5′ and 3′ ends of the heteroduplex are stabilized to prevent, e.g., “breathing” at the ends of the strands. Various means of stabilizing the ends of the heteroduplexes are contemplated by the present invention. In one embodiment, a spermine moiety is engineered into the mismatch cleavage probes at the 5′ and 3′ ends of the reference oligonucleotide. In another embodiment, a G clamp is engineered into the mismatch cleavage probes at the 5′ and 3′ ends of the reference oligonucleotide. G-clamps (Glen Research) are cytosine analogs that will selectively base pair to guanosine and will raise thermal melt temperatures significantly.

In some embodiments the mismatch cleavage probe includes a biotin moiety, enabling isolation of homoduplexes and cleavage fragments including the biotin moiety from cleavage fragments lacking the biotin fragment through binding to a SA-linked solid support, as described herein.

In certain embodiments, conditions are optionally provided to denature the cleaved heteroduplex fragments, as described.

In step E of the method, the presence of the cleaved target sequence is determined by detecting the dissociation of the first and second target identifiers, in this embodiment, by passage of nucleic acids through nanopore 170. In this embodiment, first probe fragment 160 produces a signal specific to the first target identifier because is no longer linked to second probe fragment 165. In contrast, uncleaved mismatch probe 120 produces signals specific to both the first and the second target identifiers. In this embodiment, the presence of the target sequence in the test sample is thus determined by detecting the dissociation, or uncoupling, of the two target identifiers using a nanopore-based detection system. In other embodiments, different detection systems may be used to detect other target identifier-specific signals, as disclosed further herein. An advantage of the methods of the present invention is that a “positive” signal resulting from detection of an uncoupled first signal can be confirmed by subsequent detection of the uncoupled second signal.

In some embodiments, the electronic signals are detected by passage of nucleic acids through a nanopore. Nanopores may be broadly classified into biological and synthetic types, and both types are intended to be within the scope of this invention. While alpha hemolysin (aHL) is perhaps the most studied biological nanopore to date, this and other biological nanopores may be utilized in the context of this invention, such as Mycobacterium smegmatis porin A (MspA). More recently, synthetic nanopores have been introduced using polymers, aluminum oxide, silicon dioxide, silicon nitride or other thin solid-state membranes. Nanopore-based methods and systems of detecting electronic signal are disclosed in Applicants' co-pending patent application no. s US2014/0134618 and US2017/0073740, which are herein incorporated by reference in their entirety.

In other embodiments, the electronic signals are detected by ISFET. ISFET is an ion-sensitive field-effect transistor, that is a field-effect transistor used for measuring ion concentrations in solution; when the ion concentration (such as H+, see pH scale) changes, the current through the transistor will change accordingly.

In yet other embodiments, the signals are produced by fluorescent dyes and may be detected by any suitable optical means known in the art.

In some embodiments, the methods of the present invention can further include assay controls and process controls. Assay controls, or “run” controls, include positive controls and negative controls. Control materials may be obtained commercially, prepared in-house, or obtained from other sources. Positive-control material may be purified or synthetic target nucleic acid or test samples containing the target nucleic acid. The positive control may be constructed so that it is at a concentration near the lower limit of detection of the assay. The concentration should be high enough to provide consistent positive results but low enough to challenge the detection system near the limit of detection. For multiplex systems, positive controls for each target nucleic acid are included. Positive control samples use the mismatch cleavage probe and mismatch endonucleases as the test sample that may contain the target nucleic acid. A positive control has to be tested in a separate sample from the test sample being assayed.

A blank non-target control, such as water or buffer may be used as a form of negative control. Negative controls may also be used to compensate for background signal generated by the reagents. Negative controls may be taken through the methods of the invention and contain all of the reaction reagents. A negative control may also be a test sample containing known non-target nucleic acid, such as patient specimens from individuals lacking the biomarkers of interest or non-infected individuals. A negative control assay uses the same mismatch cleavage probe and mismatch endonucleases as the test sample assay and has to occur in a separate sample from the test sample being assayed.

Internal controls, or “process” controls, refer to a control target nucleic acid that is always present in the test sample or is added to the test sample prior to nucleic acid extraction. This control verifies functionality of the sample preparation, mismatch cleavage, and detection processes. A process control must use a different mismatch cleavage probe than the target nucleic acid.

### Mismatch Cleavage Probe Configurations

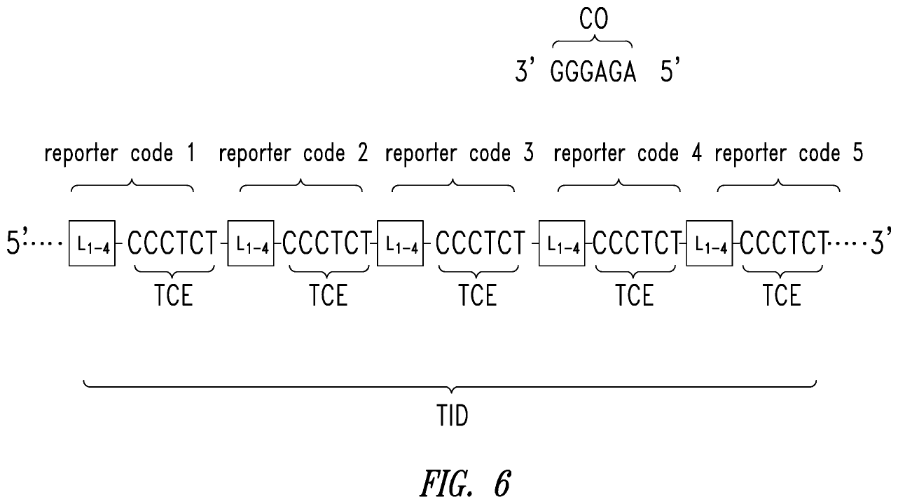

FIG. 2A is a cartoon of one embodiment of a generalized mismatch cleavage probe for detecting a single-stranded target nucleic acid of the present invention. For clarity of discussion, features illustrated in the figure are simplified and not shown to scale. Mismatch cleavage probe 200 includes oligonucleotide 210 providing a reference sequence. The reference sequence is part of the reverse complement of the single-stranded target nucleic acid and includes one or more nucleotide differences relative to the target nucleic acid. The oligonucleotide is capable of hybridizing to the target nucleic acid to form a heteroduplex, in which the heteroduplex includes one or more base pair mismatches at the positions of the one or more nucleotide differences in the reference sequence. In some embodiments, the 5′ and/or the 3′ ends of the oligonucleotide may be associated with 5′ duplex stabilizer 215A and 3′ duplex stabilizer 215B. The mismatch cleavage probe further includes first target identifier 220 linked to the oligonucleotide 5′ to the position of the one or more nucleotide differences in the reference sequence and second target identifier 230 linked to the oligonucleotide 3′ to the position of the one or more nucleotide differences in the reference sequence. Advantageously, the first and second target identifiers are capable of generating distinct and reproducibly detectable signals, e.g., electronic signals detected by passage through a nanopore. In certain embodiments, e.g., when the mismatch cleavage probes are used in connection with a nanopore-based detection system, the first and second target identifiers further include translocation control elements (TCEs), as discussed further with reference to FIG. 6. The mismatch cleavage probe may also include hydrophobic capture element (HCE) 250 associated with the first target identifier, leader sequence (LS) 260 positioned at the first end of the mismatch cleavage probe, and biotin moiety 270 positioned at the second end of the mismatch cleavage probe. However, it is to be understood that in certain embodiments of the practice of the methods of the invention, the duplex stabilizers, TCE, HCE, LS and biotin may be optional features.

FIG. 2B illustrates the two probe products generated by specific mismatch endonuclease cleavage of mismatch probe:target nucleic acid heteroduplexes. First probe product 201 includes first oligonucleotide fragment 211, 5′ duplex stabilizer 215A, first target identifier 220, hydrophobic capture element 250, and leader sequence 260. Second probe product 203 includes second oligonucleotide fragment 213, 3′ duplex stabilizer 215B, second target identifier 230, and biotin moiety 270. As described further herein, the leader sequence and the hydrophobic capture element unique to the first product enhance translocation of this product through a nanopore, whereupon the first target identifier generates a distinct electronic signal. In contrast, the second product lacks these features, which reduces its signal-producing passage through a nanopore. This uncoupling of the first and the second target identifiers through mismatch cleavage thus signals the presence of the target sequence in a test sample.

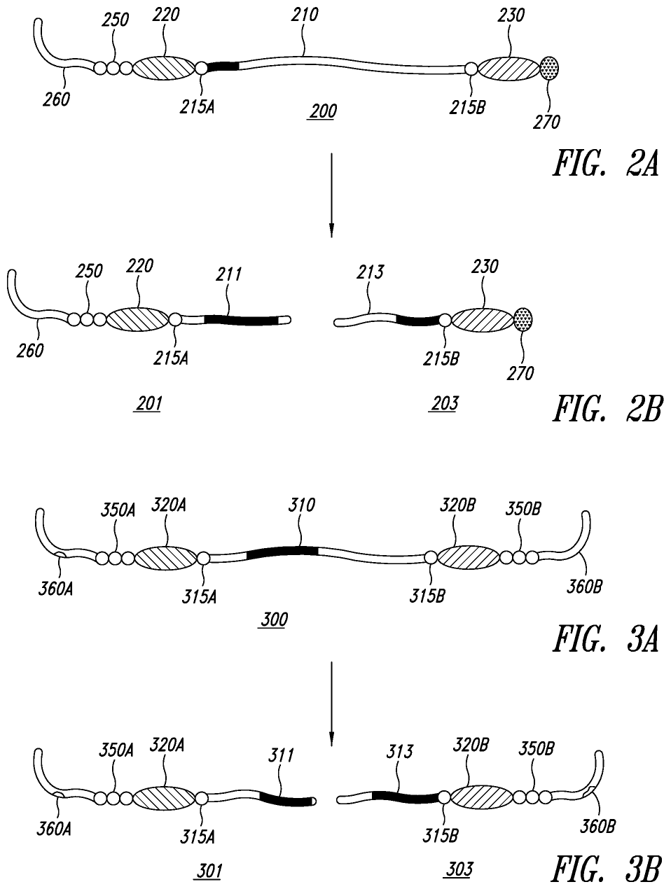

FIG. 3A is a cartoon of another embodiment of a generalized mismatch cleavage probe for detecting a single-stranded target nucleic acid of the present invention. For clarity of discussion, features illustrated in the figure are simplified and not shown to scale. Symmetrical mismatch cleavage probe 300 includes oligonucleotide 310 providing a reference sequence. The reference sequence is part of a sequence of the reverse complement of the single-stranded target nucleic acid and includes one or more nucleotide differences relative to the target nucleic acid. In some embodiments, the 5′ and/or the 3′ ends of the oligonucleotide may be associated with 5′ duplex stabilizer 315A and 3′ duplex stabilizer 315B. The oligonucleotide is capable of hybridizing to the target nucleic acid to form a heteroduplex in which the heteroduplex includes one or more base pair mismatches at the positions of the one or more nucleotide differences in the reference sequence. The mismatch cleavage probe further includes first target identifier 320A linked to the oligonucleotide 5′ to the position of the one or more nucleotide differences in the reference sequence and second target identifier 320B linked to the oligonucleotide 3′ to the position of the one or more nucleotide differences in the reference sequence. In some embodiments, the first and second target identifiers may be identical in structure but linked to the probe in opposite polarity. Advantageously, the first and second target identifiers are capable of generating distinct and reproducibly detectable electronic signals, e.g., by passage through a nanopore. The first and second target identifiers may further include translocation control elements when used in conjunction with a nanopore-based detection system. The mismatch cleavage probe may also include first hydrophobic capture element 350A associated with the first target identifier, second hydrophobic capture element 350B associated with the second target identifier, first leader sequence 360A positioned at the first end of the mismatch cleavage probe, and second leader sequence 360B positioned at the second end of the mismatch cleavage probe.

FIG. 3B illustrates the two probe products generated by specific mismatch endonuclease cleavage of symmetrical mismatch probe:target nucleic acid heteroduplexes. First probe product 301 includes first oligonucleotide fragment 311, 5′ duplex stabilizer 315A, first target identifier 320A, first hydrophobic capture element 350A, and first leader sequence 360A. Second product 303 includes second oligonucleotide fragment 313, 3′ duplex stabilizer 315B, second target identifier 320B, second hydrophobic capture element 350B, and second leader sequence 360B. As described further herein, cleavage of symmetrical mismatch probe:target nucleic acid heteroduplexes uncouples the first and second target identifiers, which are capable of independently translocating through a nanopore and generating a single distinct signal. This uncoupling of the first and the second target identifiers thus indicates the presence of the target sequence in a test sample.

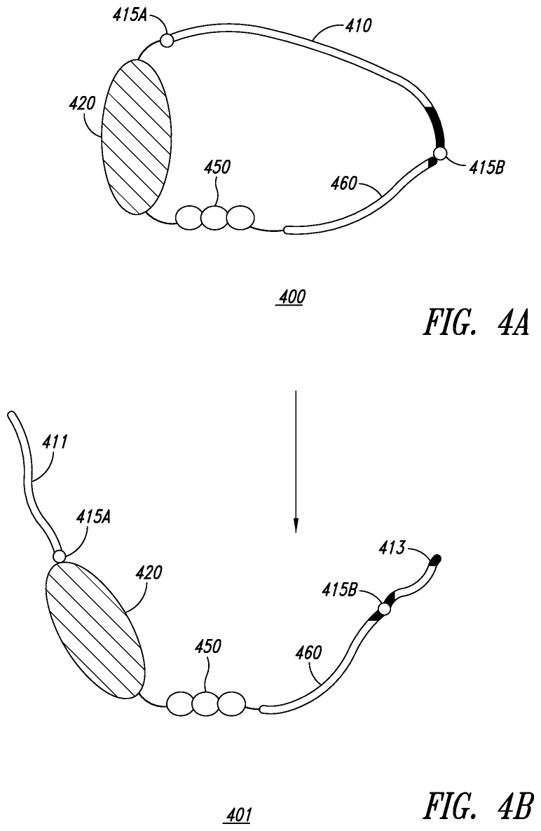

FIG. 4A is a cartoon of one embodiment of a generalized mismatch cleavage probe for detecting a single-stranded target nucleic acid of the present invention. For clarity of discussion, features illustrated in the figure are simplified and not shown to scale. Circular mismatch cleavage probe 400 includes oligonucleotide 410 providing a reference sequence. The reference sequence is part of a sequence of the reverse complement of the single-stranded target nucleic acid and includes one or more nucleotide differences relative to the target nucleic acid. In some embodiments, the 5′ and/or the 3′ ends of the oligonucleotide may be associated with 5′ duplex stabilizer 415A and 3′ duplex stabilizer 415B. The oligonucleotide is capable of hybridizing to the target nucleic acid to form a heteroduplex, in which the heteroduplex includes one or more base pair mismatches at the positions of the one or more nucleotide differences in the reference sequence. The circular mismatch cleavage probe further includes target identifier 420 linked to the 5′ end of the oligonucleotide that may include translocation control elements. Hydrophobic control element 450 is linked to the leader sequence 460, which is linked to the 3′ end of the oligonucleotide.

FIG. 4B illustrates the linear probe product generated by specific mismatch endonuclease cleavage of circular mismatch probe:target nucleic acid heteroduplexes. Linear product 401 includes first oligonucleotide fragment 411 at a first end, followed in order by 5′ duplex stabilizer 415A, target identifier 420, hydrophobic capture element 450, leader sequence 460, 3′ duplex stabilizer 415B, and second oligonucleotide fragment 413 at a second end. As described further herein, the leader sequence and the hydrophobic capture element of the linear product enhance translocation through a nanopore, whereupon the target identifier generates a distinct signal. In contrast, the uncleaved, circular product does not translocate translocation through the nanopore. Translocation of the linear product produced by mismatch cleavage through a nanopore thus signals the presence of the target sequence in a test sample.

### Target Identifiers (TID)

In certain embodiments, the target identifier constructs of the present invention each produce a unique electronic signal as they are comprised of a specific series of reporters (i.e. “codes”) sized, e.g., to block ion flow through a nanopore at different measureable levels. Specific reporter moieties can be efficiently synthesized using phosphoramidite chemistry typically used for oligonucleotide synthesis. Reporters can be designed by selecting a sequence of specific phosphoramidites from commercially available libraries. Such libraries include, but are not limited to, polyethylene glycol with lengths of 1 to 12 or more ethylene glycol units, aliphatic with lengths of 1 to 12 or more carbon units, deoxyadenosine (A), deoxycytosine (C), deoxyguanosine (G), thymine (T), abasic (Q). Table 1 below lists some representative phosphoramidites.

TABLE 1
Representative Phosphoramidites

| Phosphoramidite | Short Name | Symbol |
|---|---|---|
| Triethylene glycol | PEG-3 | X |
| Hexaethylene glycol | PEG-6 | D |
| Ethane | C-2 | L |
| Hexane | C-6 | P |
| Dodecane | C-12 | Z |
| Deoxyadenosine | dA | A |
| thymine | T | T |
| Deoxycytosine | dC | C |
| Deoxyguanosine | dG | G |
| Abasic | ab | Q |
| Spermine | Sp | S |

Each constituent phosphoramidite contributes to the net ion resistance according to its position in the nanopore, its displacement, its charge, its interaction with the nanopore, its chemical and thermal environment, and other factors. The charge on each phosphoramidite is due, in part, to the phosphate ion which has a nominal charge of −1 but is effectively reduced by counterion shielding.

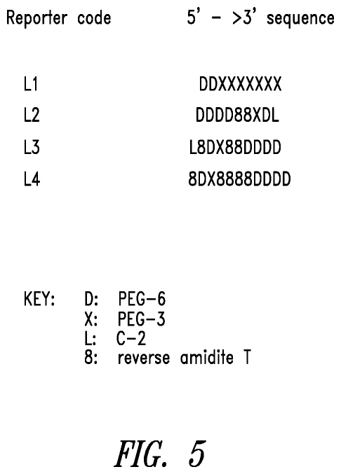

In one embodiment of the present invention, reporters are designed by choosing phosphoramidite building blocks from hexaethylene glycol (PEG-6), triethylene glycol (PEG-3), ethane (C-2), and thymine ((T). FIG. 5 shows the structure of 4 exemplary reporters, L1, L2, L3, and L4 that block ion flow in a hemolysin nanopore at four different levels, as described further with reference to Example 2.

When the detection methods of the present invention employ a nanopore-based system, the target identifiers also include a translocation control element (TCE) associated with each reporter code. TCEs provide a region of hybridization that can be duplexed to a complementary oligomer (CO) and are positioned adjacent to a reporter in the target identifier. TCEs enable translocation control by hybridization (TCH), as used herein to refer to a method to pause a nanopore translocation event by using a structure created by hybridization, which disassociates for translocation to proceed. During the methods of the present invention, as the cleaved mismatch cleavage probe fragment translocates through the nanopore, its TID enters the pore until the duplexed TCE is stalled at the pore entrance. In certain embodiments, the TCE duplex is −2.4 nm in diameter whereas the pore entrance is −2.2 nm, so the target identifier is held in the pore until the complementary strand of the duplex dissociates, whereupon translocation proceeds. Any suitable sequence or length of TCE may be used according to the present invention, provided that it enables TCH. TCH and TCEs are described in detail in Applicants' copending patent publication no. US2017/0073740, which is disclosed herein in its entirety.

A key feature of the detection methods of the present invention is the ability to multiplex the detection assay. In these embodiments, a plurality of TIDs is designed in which each individual TID includes a multimeric sequence of reporter codes, wherein each reporter code “monomer” is associated with a TCE. By multimeric sequence of reporter codes is meant at least two reporter code monomeric units in series. However, the invention is not intended to be limited to any particular range of reporter code monomeric units comprising the TID nor in the number of unique reporter codes (i.e. “levels”) occupying each position in the sequence of monomeric units. One constraint in designing a TID is that adjacent positions in the multimer cannot be occupied by the same reporter code. One exemplary embodiment is illustrated in FIG. 6, in which each of the plurality of TIDs designed for a multiplex detection assay includes a multimer of five reporter codes in series. In this embodiment, each position in the series is occupied by one of four unique reporter codes (i.e., four levels for each position). In this embodiment, a pool of around 768 unique TIDs is produced. As depicted, from the 5′ end to the 3′ end, the TID includes a multimer of five reporter codes, each associated with a TCE with the sequence, CCCTCT. In this embodiment, the four levels of reporter codes include the four unique reporter codes, L1, L2, L3, and L4 illustrated in FIG. 5A. Also depicted in FIG. 6 is the complementary oligonucleotide (CO) that is used for TCH.

### Duplex Stabilizers

As disclosed herein, a key feature of the methods of the present invention is the reliable specificity of mismatch endonuclease activity. The inventors have surprisingly and advantageously discovered that the specificity of mismatch cleavage can be enhanced when at least one duplex stabilizer moiety is linked to the 5′ and/or 3′ ends of reference oligonucleotide in the mismatch cleavage probes. Without being bound by theory, it is hypothesized that inclusion of such duplex stabilizers prevents the ends of the stands comprising the duplexed nucleic acid from “breathing”, or transiently dissociating. Reduction of this dynamic movement thus may provide a more stable heteroduplex substrate for cleavage by mismatch endonucleases. In other words, “locking” the ends of the heteroduplexes may enhance mismatch endonuclease cleavage. Non-limiting examples of duplex stabilizers include spermine and G-clamps.

### Hydrophobic Capture Elements (HCE) and Leader Sequences (LS)

The hydrophobic capture elements and leader sequences of the present invention improve the probability of interaction between a mismatch cleavage probe or fragment thereof and a nanopore by capturing the probe of probe fragment on a surface comprising the nanopore. The captured mismatch cleavage probe or fragment thereof, the nanopore, or both, are able to move relative to each other along the surface. In this way, the volume occupied by the mismatch cleavage probe or fragment thereof and the nanopore is dramatically reduced compared, for example, to a probe in a volume of solution that is in contact with the surface. By confining the mismatch cleavage probe or fragment thereof and nanopore in this manner—also referred to herein as “concentrating” the probe—the probability of interaction between the probe and the nanopore is significantly increased. Such increased concentration leads to significantly enhanced translocation of the mismatch cleavage probe or fragment thereof, through the nanopore. According the present invention, the mismatch cleavage probe or fragment thereof includes one or more target identifiers.

The hydrophobic capture element associates with the hydrophobic domain of the surface. As used herein, associated means that the hydrophobic capture element of the mismatch cleavage probe or fragment thereof and the hydrophobic domain of the surface cause the probe to remain joined to the surface, while also permitting the captured probe to move along the hydrophobic domain of the surface to bring the target molecule in proximity with the nanopore. Such hydrophobic-hydrophobic interaction is mostly an entropic effect associated with disruption of highly dynamic hydrogen bonds between water molecules and nonpolar substances. The strength of hydrophobic interactions depends on temperature, as well as the shape and number of carbon atoms on the hydrophobic compound.

Materials that comprise the hydrophobic capture element include, but are not limited to, linear and branched aliphatic chains, lipids, fatty acids, DBCO, cholesterol, fluorinated polymers, apolar polymers, steroids, polyaromatic hydrocarbons, hydrophobic peptides, and hydrophobic proteins. This may also include phase transition polymers that can switch from hydrophilic to hydrophobic states under thermal or other environmental change.

Once captured by the surface, the leader portion of the mismatch cleavage probe or fragment thereof is capable of interacting with the nanopore in a manner that promotes interaction of the probe (or target portion thereof) with the nanopore. Such interaction includes, for example, complete or partial translocation through the nanopore. Typically, the leader is not hydrophobic, and in one embodiment is a hydrophilic (charged) polymer of low mass to allow interaction with the nanopore when the nanopore and the leader of the mismatch cleavage probe or fragment thereof are in close proximity. As mentioned above, the captured mismatch cleavage probe or fragment thereof, the nanopore, or both, are capable of movement relative to each other along the surface.

The leader length that extends beyond the hydrophobic capture element may also be modified for interaction with the nanopore. To this end, the leader should be of a sufficient length such that its capture in the nanopore exerts enough force to uncouple the mismatch cleavage probe or fragment thereof from the bilayer or, depending on the embodiment, unlink the leader/target portion from the hydrophobic capture element. The leader should carry electrostatic charge to promote interaction with the nanopore under an applied electric potential. A nucleic acid is typically anionic and the leader would typically also be anionic. The leader is typically a single linear polymer, but may have two or more linear polymer portions to help improve nanopore interaction, and should also be able to translocate the nanopore so the target molecule can then engage. Leader materials can be synthesized from many anionic, cationic or neutral polymers and may be made of combinations of materials such as (but not limited to) heterogenous or homogeneous polynucleotides, polyethylene glycol, polyvinyl alcohol, polyphosphates, poly(vinylphosphonate), poly(styrenesulfonate), poly(vinylsulfonate), polyacrylate, abasic deoxyribonucleic acid, abasic ribonucleic acid, polyaspartate, polyglutamate, polyphosphates, and the like. For example, a representative leader may comprise PEG-24 and/or poly-A₁₂. In another specific embodiment, the hydrophobic capture element is a C48 aliphatic hydrophobic group and the leader is polyA₂₄ oligomer that acts as a hydrophilic polyanionic leader.

Hydrophobic capture elements and leader sequences are described in detail in Applicants' copending patent publication no. US2014/0134618, herein disclosed by reference in its entirety.

### Signal Amplification During Detection by Mismatch Cleavage

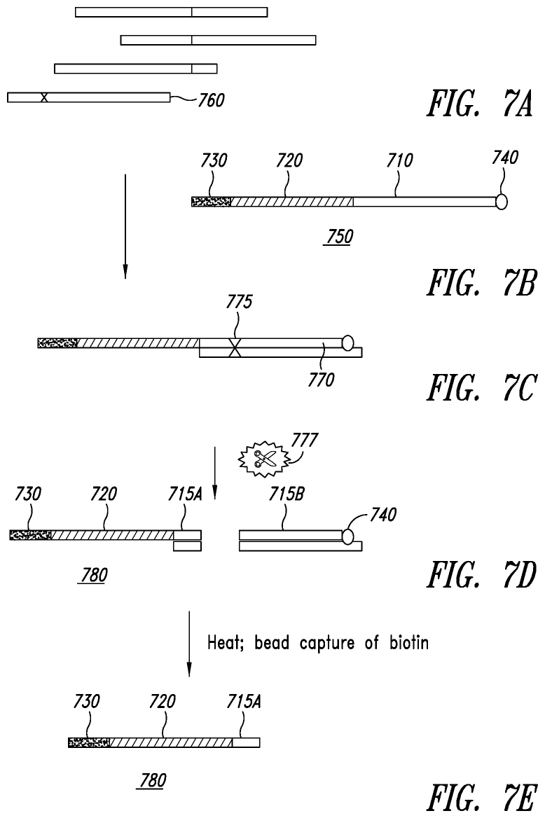

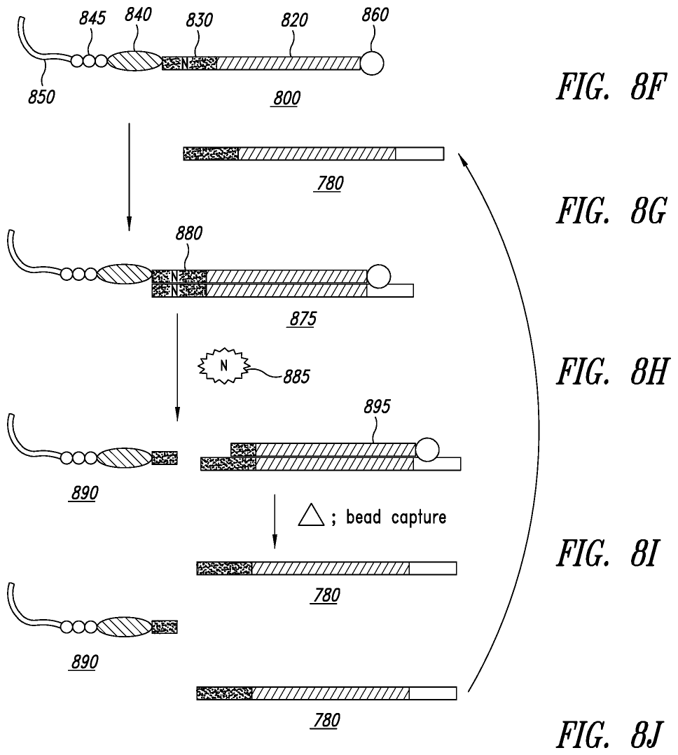

In certain embodiments, the methods of the present invention may be modified in order to amplify the signal generated by cleavage of a mismatch cleavage probe. The signal amplification method of the present invention includes two different stages, each utilizing a distinct probe: 1) a mismatch cleavage stage utilizing a mismatch amplifier probe and 2) a signal amplification stage utilizing an amplification code probe. FIG. 7 summarizes the methods of one embodiment of the mismatch cleavage stage. Steps A-D of the method are similar to those discussed with reference to FIG. 1. Briefly, in step A, a test sample is provided that contains a mixture of denatured nucleic acids, including target nucleic acid 760. As disclosed herein, the target nucleic acid includes at least one polymorphism or mutation relative to a reference signal, denoted in the cartoon illustration by the symbol, “x”.

In step B, mismatch amplifier probe 750 is provided that includes reference oligonucleotide 710, first hybridization oligonucleotide 720, first nickase recognition oligonucleotide 730, and biotin moiety 740. From a first end to a second end of the probe, the sequence of these elements is: first nickase recognition oligonucleotide, first hybridization oligonucleotide, reference oligonucleotide, and biotin moiety. As discussed with reference to FIG. 2, the sequence of the reference oligonucleotide is part of the reverse complement of the single-stranded target nucleic acid and includes one or more nucleotide differences relative to the target nucleic acid. The oligonucleotide is capable of hybridizing to the target nucleic acid to form a heteroduplex, in which the heteroduplex includes one or more base pair mismatches at the positions of the one or more nucleotide differences relative to the reference sequence. In some embodiments, the 5′ and/or the 3′ ends of the oligonucleotide may be associated with one or more duplex stabilizers, as disclosed herein. The hybridization oligonucleotide is designed to hybridize and form a stable duplex with a reverse complement sequence, which is a feature of the code probe, discussed with reference to FIG. 8. The hybridization oligonucleotide may be any length or sequence suitable to form such a stable duplex. The first nickase recognition oligonucleotide includes one strand of a recognition site for a nickase endonuclease. Nickase endonucleases refer to enzymes that recognize a specific sequence in a double-stranded nucleic acid, and cut one strand at a specific location relative to the recognition sequence, thereby giving rise to single-stranded breaks in the double-stranded nucleic acid. Non limiting examples of nickases include Nb.BbvCI, Nb.BsmI, Nb.BsrDI, Nb.Btsl, NtAIwI, Nt.BbvCI, Nt.BsmAI, Nt.BspQI, NtBstNBI, and Nt.CviPII. Conditions for using nickase endonucleases to generate single-stranded breaks in double-stranded nucleic acids are well known in the art.

In step C of the method, the mismatch amplifier probe is mixed with the test sample under annealing conditions to allow formation of heteroduplex 770 between the mismatch amplifier probe and the target sequence, in which the heteroduplex contains at least one single base pair mismatch 775.

In step D of the method, the mixture is treated with at least one endonuclease 777 that is capable of cleaving the mismatched base pair in the heteroduplex. Mismatch cleavage of the heteroduplex generates cleaved amplifier probe 780 that includes the first nickase recognition oligonucleotide 730 and the first hybridization oligonucleotide 720, as well as first reference oligonucleotide fragment 715A. A second probe fragment includes second reference oligonucleotide fragment 715B and the biotin moiety 740.

In step E of the method, the double-stranded nucleic acid fragments are denatured, e.g., by heating the sample, and the second probe fragment is removed from the mismatch amplifier probe by, e.g., SA-coated beads. The cleaved amplifier probe, also referred to simply as the “amplifier probe”, is used in the second stage, described below.

The methods of the signal amplification stage of the present invention are summarized with reference to FIG. 8. In step F of the method, amplification code probe 800 is provided that includes second hybridization oligonucleotide 820, second nickase recognition oligonucleotide 830, target identifier 840, hydrophobic capture element 845, leader sequence 850 and streptavidin moiety 860. The target identifier, hydrophobic capture element, and the leader sequence are associated with a first end of the nickase recognition oligonucleotide, while the hybridization oligonucleotide and the streptavidin are associated with a second end of the nickase recognition oligonucleotide. The sequence of the second hybridization oligonucleotide includes the reverse complement of the first hybridization oligonucleotide of the mismatch amplifier probe, while the second nickase recognition oligonucleotide includes the reverse complement of the first nickase recognition oligonucleotide of the mismatch amplifier probe. The second nickase recognition oligonucleotide of the code probe is the strand cleaved by nickase when duplexed with its complement, herein illustrated by the symbol “N”. As disclosed herein, the target identifier is capable of generating a distinct and reproducibly detectable electronic signal, e.g., by passage through a nanopore and further include translocation control elements, while the leader sequence and hydrophobic capture element provide features to enhance the rate of translocation through a nanopore.

In step G of the method, conditions are provided to hybridize the mismatch amplifier probe 780, generated during the mismatch cleavage stage, to the code probe to form double-stranded polynucleotide 875 that includes double-stranded nickase site 880. Hybridization is primarily mediated by annealing of the first hybridization oligonucleotide to the second hybridization oligonucleotide.

In step H of the method, the double-stranded nickase site is contacted with nickase endonuclease 885 to cleave the second nickase recognition oligonucleotide and release cleaved code probe 890. As disclosed herein, when duplexed with its reverse complement to provide a cleavage substrate, the nickase recognition oligonucleotide of the mismatch amplifier probe remains intact, while the reverse complement strand is cleaved by the nickase endonuclease. The cleaved code probe includes the target identifier, hydrophobic capture element, and leader sequence, while probe fragment 895 include the hybridization oligonucleotide and the streptavidin moiety. Thus, the cleaved code probe includes all the features necessary for production of a specific electronic signal by translocation through a nanopore.

In step I of the method, the sample is denatured, e.g., by heating to release the uncleaved amplifier probe 780. In certain embodiments, the nickase enzyme of step H is an engineered variant with increased thermostability that is capable of maintaining endonuclease activity during repeated cycles of step I.

In step J of the method, the amplifier probe is recycled a plurality of times through steps G-H to provide a plurality of cleaved code probes. Following the amplification stage, the plurality of code probes is electronically measured by, e.g., a nanopore-based detection system.

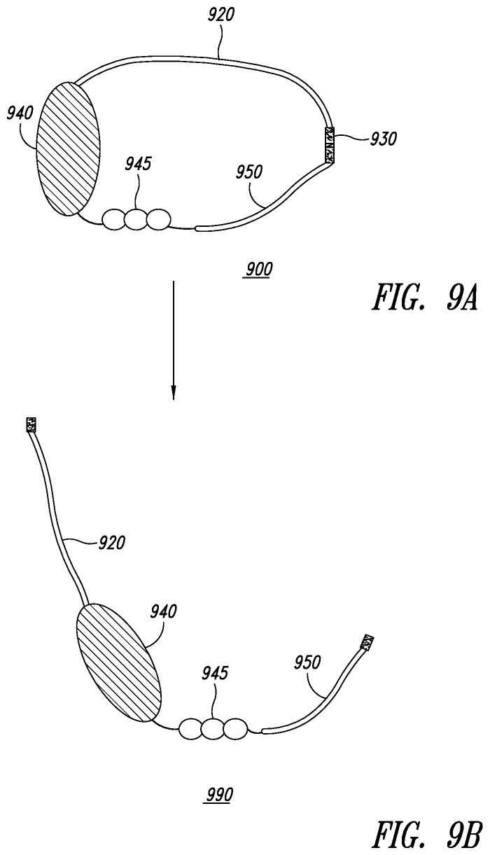

An alternative amplification code probe configuration is illustrated in FIG. 9A. In this embodiment, circular amplification code probe 900 includes second hybridization oligonucleotide 920, second nickase site 930, target identifier 940, hydrophobic capture element 945, and leader sequence 950. The circular configuration of this amplification code probe prevents translocation through a nanopore. In contrast, linearization of the circular amplification code probe into linear configuration 990, as depicted in FIG. 9B, enables translocation through a nanopore. Linearization is mediated by nickase cleavage of the second nickase site when the amplifier probe is hybridized to the circular amplification code probe to create a double-stranded nickase recognition site, as described with reference to FIG. 8.

One skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3rd edition), Cold Spring Harbor Press, Cold Spring Harbor, N.Y. (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, Pa., 18th edition (1990). These texts can, of course, also be referred to in making or using an aspect of the disclosure.

### EXAMPLES

### Example One

Endonuclease-Mediated Mismatch Cleavage of a DNA Heteroduplex

This Example demonstrates endonuclease-mediated cleavage of a DNA heteroduplex possessing a single base pair mismatch, while a perfectly paired DNA homoduplex remains resistant to cleavage. For this experiment, single-stranded oligonucleotides representing the “match” and “mismatch” target sequences were hybridized with a single-stranded oligonucleotide probe. The sequence of the match oligonucleotide was as follows: 5′ GATTTTAATCACAATTCCACATGACGGGAGCCGGAAGCATAAAGTGAACTA G3′; the sequence of the mismatch oligonucleotide was as follows: 5′ GATTTTAATCACAATTCCACATGACGCGAGCCGGAAGCATAAAGTGAACTA G3′; with the position of the polymorphism in the target sequence indicated in bold. The sequence of the mismatch oligonucleotide probe was as follows: 5′WCTAGTTCACTTTATGCTTCCGGCTCCCGTCATGTGGAATTGTGATTAAAA TC3′. The mismatch probe was designed with features to reduce non-specific cleavage and increase stability of the heteroduplex, including G-clamps located at the 5′ and 3′ ends of the oligonucleotide (indicated by the italicized letter, “C”) and 2′OMe nucleotide analogs (identified by underscoring). The position of the polymorphism in the target sequence is identified by the letter “C” in bold. The “W” symbol in the probe represents the detection label, SIMA (HEX) fluor.

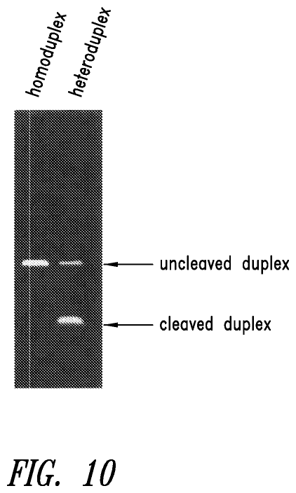

For the hybridization reaction, 2 pmol of probe oligonucleotide was mixed with 2.5 pmol of match or mismatch target oligonucleotide in hybridization buffer composed of 10 mM Tris-HCl, pH 8.8; 20 mM MgCl₂, and 125 mM KCl. The temperature of the hybridization reaction was ramped-down from an initial 95° C. (for 10 minutes) to 25° C. in steps of ten degrees with holds of one minute at each step. The cooled homoduplex and heteroduplex samples were then treated with Surveyor® endonuclease (IDT) in the hybridization buffer and reaction products were analyzed by gel electrophoresis. As shown in FIG. 10, the perfectly paired homoduplex sample was resistant to endonuclease-mediated cleavage, while the heteroduplex sample (in which the duplex contains a single base pair mismatch) was cleaved by the endonuclease to generate the smaller cleavage fragments indicated by the arrow.

### Example 2

### Detection of Target Identifiers in a A-Hemolysin Nanopore

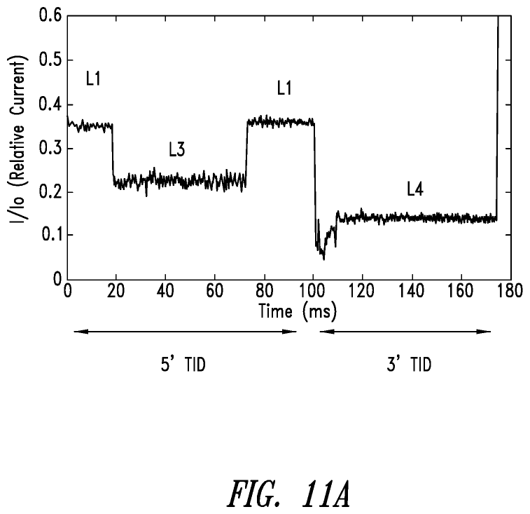

FIGS. 11A and 11B are time traces that record the current measurement caused by two different mismatch cleavage (MMC) probes, MMC probe A and MMC probe B, passing through a α-Hemolysin nanopore. These were recorded with a 100 kHz bandwidth filter on an Axopatch 200B amplifier, and demonstrate reporter resolution <25 us/reporter. The general features of MMC probes A and B are illustrated in FIG. 2A and each includes both a 5′ and a 3′ target identifier (TID). The 5′ TID of MMC probe A is composed of a series of three reporter codes: L1-L3-L1 (as discussed with reference to FIG. 5) while the 3′ TID includes a single reporter code, L4. The 5′ TID of MMC probe B is composed of a series of three reporter codes: L2-L4-L2, while the 3′ TID includes a single reporter code L3. The traces show the four distinguishable current levels reproducibly produced by each of the four reporter codes, L1, L2, L3, and L4 with the trace produced by MMC probe A shown in FIG. 11A and the trace produced by MMC probe B shown in FIG. 11B. The signals observed between the 5′ and 3′ TID signals represent background generated by the oligonucleotide probe.

All of the U.S. patents, U.S. patent application publications, U.S. patent applications, foreign patents, foreign patent applications and non-patent publications referred to in this specification and/or listed in the Application Data Sheet, including but not limited to, U.S. Provisional Patent Application No. 62/541,285 are incorporated herein by reference, in their entirety. Such documents may be incorporated by reference for the purpose of describing and disclosing, for example, materials and methodologies described in the publications, which might be used in connection with the presently described invention.

1. A method for determining at least one mutation or a polymorphism in a single molecule of a target sequence of a polynucleotide relative to a reference sequence of the polynucleotide comprising:
(a) providing a test sample comprising a plurality of single-stranded polynucleotides;
(b) providing a mismatch cleavage probe comprising:
i. an oligonucleotide, wherein the oligonucleotide comprises a reference sequence, wherein the reference sequence comprises a sequence of the reverse complement of the single-stranded target nucleic acid and contains one or more nucleotide differences relative to the target nucleic acid, wherein the oligonucleotide is capable of hybridizing to the target nucleic acid to form a heteroduplex, wherein the heteroduplex comprises one or more base pair mismatches;
ii. a first target identifier linked to the oligonucleotide 5′ to the position of the one or more nucleotide differences; and
iii. a second target identifier linked to the oligonucleotide 3′ to the position of the one more nucleotide differences;
wherein the first and second target identifiers are capable of generating distinct and reproducibly detectable signals
(c) mixing the test sample with the mismatch cleavage probe under annealing conditions to form heteroduplexes between the mismatch cleavage probe and the target sequence;
(d) contacting the heteroduplexes with a cleavage factor, wherein the cleavage factor is capable of cleaving mismatched bases in the heteroduplexes, wherein cleavage of the heteroduplex dissociates the first and second target identifiers of the mismatch cleavage probe;
(e) optionally providing conditions to denature the heteroduplexes; and
(f) determining the presence of the cleaved target sequence by electronically detecting the dissociation of the first and second target identifiers.

2. The method of claim 1, wherein the cleavage factor is an endonuclease.

3. The method of claim 1, wherein the test sample comprises cell-free DNA.

4. The method of claim 1, wherein the method is multiplexed by providing a plurality of pooled mismatched cleavage probes in step (b) to determine at least one mutation in a plurality of target sequences.

5. The method of claim 4, wherein the plurality of target sequences comprises a plurality of biomarkers.

6. The method of claim 4, wherein the plurality of target sequences comprises target sequences from a plurality of test subjects.

7. The method of claim 4, wherein the plurality of target sequences comprises a plurality of fragments comprising the entire sequence of one or more test genes.

8. The method of claim 1, further comprising a polishing step to reduce the concentration of damaged nucleic acids in the test sample damage prior to the step of mixing the test sample with the mismatch cleavage probe.

9. The method of claim 1, further comprising a polishing step to reduce the concentration of damaged mismatch cleavage probes prior to the step of mixing the test sample with the mismatch cleavage probe.

10. The method of claim 1, further comprising a step to isolate the heteroduplexes by binding to an immobilized MutS protein prior to the step of contacting the heteroduplexes with a mismatch endonuclease.

11. The method of claim 2, further comprising a step to optimize conditions for mismatch cleavage prior to the step of contacting the heteroduplexes with the endonuclease.

12. The method of claim 2, wherein the endonuclease is a variant engineered to increase specificity for mismatched base pairs.

13. The method of claim 1, wherein the mismatch cleavage probe comprises at least one duplex stabilizer moiety at an end of the reference oligonucleotide.

14. The method of claim 1, wherein the step of determining the presence of the cleaved target sequence comprises passage of the cleaved mismatch cleavage probes through a nanopore to detect electronic signals.

15. The method of claim 1, further comprising one or more controls selected from the group consisting of positive controls, negative controls, and process controls.

16. A mismatch cleavage probe for detecting a single molecule single-stranded target nucleic acid in a sample comprising:
(a) an oligonucleotide, wherein the oligonucleotide comprises a reference sequence, wherein the reference sequence comprises a sequence of the reverse complement of the single-stranded target nucleic acid and contains one or more nucleotide differences relative to the target nucleic acid, wherein the oligonucleotide is capable of hybridizing to the target nucleic acid to form a heteroduplex, wherein the heteroduplex comprises one or more base pair mismatches;
(b) a first target identifier linked to the oligonucleotide 5′ to the position of the one or more nucleotide differences, and
(c) a second target identifier linked to the oligonucleotide 3′ to the position of the one more nucleotide differences;
wherein the first and second target identifiers are capable of generating distinct and reproducibly detectable signals.

17. The mismatch cleavage probe of claim 16, wherein the distinct and reproducibly detectable signals are electronic signals.

18. The mismatch cleavage probe of claim 17, wherein the first and second target identifiers comprise translocation control elements.

19. The mismatch cleavage probe of claim 18, which further comprises a hydrophobic capture element and a leader sequence associated with the first target identifier and a biotin moiety associated with the second target identifier.

20. The mismatch cleavage probe of claim 16, which further comprises a first hydrophobic capture element and a first leader sequence associated with the first target identifier and a second hydrophobic capture element and a second leader sequence associated with the second target identifier.

21. The mismatch cleavage probe of claim 18, wherein the target identifiers comprise a plurality of unique codes, wherein each individual code is associated with a translocation control element.

22. The mismatch cleavage probe of claim 21, wherein the target identifiers comprise from around 2 to around 10 codes.

23. The mismatch cleavage probe of claim 22, wherein the sequence of the each code is selected from the group consisting of: DDXXXXXXX, DDDD88XDL, L8DX88DDDD, and 8DX8888DDDD, wherein D is PEG-6, X is PEG-3, 8 is reverse amidite T, and L is C2.

24. The mismatch cleavage probe of claim 16, further comprising a duplex stabilizer associated with at least one end of the reference oligonucleotide.

25. The mismatch cleavage probe of claim 24, wherein the duplex stabilizer is a spermine or a G-clamp moiety.

26. The mismatch cleavage probe of claim 16, wherein the sequence of the reference oligonucleotide comprises the wild-type allele of a tumor biomarker.

27. The mismatch cleavage probe of claim 16, wherein the sequence of the reference oligonucleotide comprises a sequence from a pathogenic microorganism.

28. A circular mismatch cleavage probe for detecting a single molecule target nucleic acid in a sample comprising:
(a) an oligonucleotide, wherein the oligonucleotide comprises a reference sequence, wherein the reference sequence comprises a sequence of the reverse complement of the single-stranded target nucleic acid and contains one or more nucleotide differences relative to the target nucleic acid, wherein the oligonucleotide is capable of hybridizing to the target nucleic acid to form a heteroduplex, wherein the heteroduplex comprises one or more base pair mismatches;
(b) a target identifier linked to the 5′ end of the oligonucleotide, wherein the target identifier comprises a translocation control element and wherein the target identifier is capable of generating a distinct and reproducibly detectable signal upon passage through a nanopore; and
(c) a leader sequence associated with a hydrophobic capture element, wherein the hydrophobic capture element is linked to the target identifier and the leader sequence is linked to the 3′ end of the oligonucleotide;
wherein the circular mismatched cleavage probe is not capable of passage through a nanopore, wherein cleavage of the oligonucleotide linearizes the mismatch cleavage probe, and wherein the linear mismatch cleavage probe is capable of passage through a nanopore.

29. A method for amplifying a signal indicating at least one mutation or a polymorphism in a target sequence of a polynucleotide relative to a reference sequence of the polynucleotide comprising:
(a) (Original) a mismatch cleavage stage, wherein the mismatch cleavage stage comprises contacting the target sequence with a mismatch amplifier probe and a mismatch endonuclease to produce a cleaved amplifier probe;
(b) iterative rounds of a signal amplification stage, wherein a single round of the signal amplification stage comprises contacting the amplifier probe with a pool of amplification code probes and a nickase enzyme to produce a cleaved amplification code probe capable of producing a distinct and reproducible signal upon passage through a nanopore.

30. The method of claim 29, wherein the mismatch cleavage stage comprises the steps of:
(a) providing a test sample comprising a plurality of denatured polynucleotides;
(b) providing a mismatch amplifier probe comprising a reference oligonucleotide, a first hybridization oligonucleotide, and first nickase recognition oligonucleotide, and a biotin moiety;
(c) mixing the test sample with the mismatch amplifier probe under annealing conditions to form heteroduplexes between the mismatch amplifier probe and a target sequence;
(d) contacting the heteroduplexes with an endonuclease capable of cleaving mismatched bases in the heteroduplex, wherein cleavage of the heteroduplex releases an amplifier probe comprising the first hybridization oligonucleotide and the first nickase recognition oligonucleotide; and
(e) removing the biotin moiety and associated nucleic acids from the test sample.

31. The method of claim 29, wherein the signal amplification stage comprises the steps of:
(f) providing a pool of amplification code probes, wherein the amplification code probes comprise a second hybridization oligonucleotide, a second nickase recognition oligonucleotide, a target identifier, a hydrophobic capture element, a leader sequence, and a streptavidin moiety;
(g) providing conditions to hybridize the amplification code probes of step (d) to the amplifier probe of claim 28 to form a double-stranded nucleic acid comprising a double-stranded nickase site;
(h) contacting the double-stranded nickase site with a nickase endonuclease to cleave the second nickase recognition oligonucleotide and release a cleaved amplification code probe;
(i) heating the sample to release the uncleaved amplifier probe; and
(j) recycling the amplifier probe a plurality of times through steps G through I to provide a plurality of cleaved amplification code probes.

32. A mismatch amplifier probe for amplifying a signal indicating at least one mutation or a polymorphism in a target sequence of a polynucleotide relative to a reference sequence of the polynucleotide comprising:
(a) an oligonucleotide, wherein the oligonucleotide comprises a reference sequence, wherein the reference sequence comprises a sequence of the reverse complement of the single-stranded target nucleic acid and contains one or more nucleotide differences relative to the target nucleic acid, wherein the oligonucleotide is capable of hybridizing to the target nucleic acid to form a heteroduplex, wherein the heteroduplex comprises one or more base pair mismatches;
(b) a first hybridization oligonucleotide;
(c) a first nickase recognition oligonucleotide; and
(d) a biotin moiety.

33. An amplification code probe for amplifying a signal indicating at least one mutation or a polymorphism in a target sequence of a polynucleotide relative to a reference sequence of the polynucleotide comprising:
(a) second hybridization oligonucleotide, wherein the sequence of the second hybridization oligonucleotide comprises the reverse complement of the sequence of the first hybridization oligonucleotide;
(b) a second nickase recognition oligonucleotide, wherein the sequence of the second nickase recognition oligonucleotide comprises the reverse complement of the sequence of the first nickase recognition oligonucleotide, and wherein the second nickase recognition oligonucleotide is capable of being cleaved by a nickase endonuclease;
(c) a target identifier;
(d) a hydrophobic capture element;
(e) a leader sequence; and
(f) a streptavidin moiety.

34. A circular amplification code probe for amplifying a signal indicating at least one mutation or a polymorphism in a target sequence of a polynucleotide relative to a reference sequence of the polynucleotide comprising:
(a) second hybridization oligonucleotide, wherein the sequence of the second hybridization oligonucleotide comprises the reverse complement of the sequence of the first hybridization oligonucleotide;
(b) a second nickase recognition oligonucleotide linked to the 3′ end of the second hybridization oligonucleotide, wherein the sequence of the second nickase recognition oligonucleotide comprises the reverse complement of the sequence of the first nickase recognition oligonucleotide, and wherein the second nickase recognition oligonucleotide is capable of being cleaved by a nickase endonuclease;
(c) a target identifier linked to the 5′ end of the second hybridization oligonucleotide;
(d) a hydrophobic capture element linked to the 5′ end of the target identifier; and
(e) a leader sequence linked to the 5′ end of the hydrophobic capture element and the 3′ end of the second nickase recognition oligonucleotide.


SEQUENCE LISTING (3 sequences)

SEQ ID NO: 1 - DNA, 52 bp, Artificial Sequence
  Synthetic match oligonucleotide
gattttaatcacaattccacatgacgggagccggaagcataaagtgaactag

SEQ ID NO: 2 - DNA, 52 bp, Artificial Sequence
  Synthetic mis-match oligonucleotide
gattttaatcacaattccacatgacgcgagccggaagcataaagtgaactag

SEQ ID NO: 3 - DNA, 50 bp, Artificial Sequence
  Synthetic mismatch oligonucleotide probe
  modified_base 1: G-clamps and detection label, SIMA (HEX) fluor
  modified_base 50: G-clamp
tagttcactttatgcttccggctcccgtcatgtggaattgtgattaaaat